(19) United States
(12) Patent Application Publication
(10) Pub. No.: US 2011/0054782 A1
(43) Pub. Date: Mar. 3, 2011

(54) Method and apparatus of measuring and analyzing user movement

(76) Inventor: Keaka K. A. Kaahui, Lahaina, HI (US)

(21) Appl. No.: 12/461,878

(22) Filed: Aug. 27, 2009

(51) Int. Cl. G06F 19/00; A61B 5/22; G06F 15/00; G01C 21/00

(52) U.S. Cl. 701/208; 73/379.01; 73/865.4; 702/141

(57) ABSTRACT

A method and apparatus or measuring a person's movement via a plurality of sensors is enclosed. One example method may include measuring at least one rotational value during the movement via a first sensor. Additional measurements may include measuring at least one linear value during the movement via a second sensor, and measuring a force applied from a portion of the person's body via a third sensor. The measurements may be used to generate a user interface display of the person's movement on an electronic device.

### TECHNICAL FIELD OF THE INVENTION

This invention relates to a method and apparatus of determining and analyzing a user's movement via a device worn on the user's body that measures certain changes from the user's original position and provides a result of the user's change in position.

### BACKGROUND OF THE INVENTION

In the field of athletics, the accuracy of the athletes' movements are pertinent to his or her performance and success. Consistency also plays a pertinent role in the athletes' success. For example, if an athlete can consistently replicate an accurate performance than he/she may be paid millions of dollars to swing a golf club, throw a football, swing a baseball bat, shoot a basketball, etc. As a result, athletes' and their instructors spend hours examining the fine details of hand motion along with arm motion during a particular swinging or throwing motion.

Of particular interest to athletes and instructors is what is often referred to as wrist “wind-up/cock” and “release.” This motion is also known as “flicking” your wrist while throwing a football or shooting a basketball. In addition, “wrist release” is a more common term in sports using a club, bat, or racket.

Often, good athletes are said to have extraordinary “timing” or “feel”, but this innate sense of “timing” in most sports like golf, baseball, football, basketball, etc. may be broken down into the physical motion of releasing your wrist at the proper time during a larger body movement of the arm and/or legs. The consistency of when a wrist cock and release action happens, at what force it happens, along with the orientation of where on the wrist it happens during a athletic swing or movement of the arm separates the good athletes from the best.

Wrist cock and release is essential to shooting a basketball, throwing a football, swinging a golf club, etc., none of which can be done satisfactorily without some type of wrist movement. In each of these scenarios it is virtually impossible to get successful results without the wrist release being a major factor in the player's movement.

Generally, it can be said that humans are dexterous beings. Our feet and legs take us to where we want to go, our torso sets us to a general alignment for the task at hand, our arms enable us to be in a proximity of the task, but yet it is our hands and only our hands along with a wrist that permits us fill in the details and actually complete the task at hand. Athletes' tasks tend to be more aggressive and difficult, thus requiring more abrupt, powerful and precision oriented movements.

There exists many athletic motion analysis hardware and/or software packages available throughout the world today. For instance, video analysis is popular when analyzing a player's golf swing, and is used at golf academies and sports instructional institutions as well as at colleges across the world. Although this approach to player's analysis may be accurate and dependable, a few shortcomings exist with video analysis methods in sports training. For instance, one concern is the inability to create a real world environment to actually measure the player's swing and movement. Since most video analysis procedures require multiple fixed camera positions, special lighting to obtain proper imaging sequences, and high speed computers and monitors, the execution of the training procedure is not likely to occur anywhere near a playing field or golf course.

It is not feasible to analyze a player's motion at a measurement facility and expect that motion to be the same during the game (i.e. playing 18 holes on a golf course, swinging at baseball pitches on the diamond, and throwing a football on the field). In addition, the use of such equipment requires an expert technician as well as a coach to interpret what has been analyzed versus what needs to be adjusted in the athlete's motion. Such methods of analyzing a player's movement can be expensive, especially since repeated sessions are often needed to see improvement in the player's movements. Furthermore, the accessibility to video analysis facilities and coaching are limited to those with either extraordinary talent, or the monetary means to purchase such a service.

Other types of conventional athletic motion devices include a series of sensor microchips, processors, and wireless transmitters installed inside or on sporting equipment, such as, golf clubs, bats, rackets, hockey sticks, and footballs that detect the motion of the equipment. These devices require different types of calibration and are subject to inaccuracy that results from varying types of sports equipment and different sports activities. For example, there is an inherent probability that all sporting equipment wears down, and is usually reduced to waste each new season or when new technology of materials and design exceed the previous year's model.

Yet further devices may include a sport movement analyzer and training device that may be worn on the wrist. However, these devices are expensive and have many components. Additionally, the inability to detect wrist-release further limits the accuracy of analyzing the player's movement.

### SUMMARY OF THE INVENTION

One embodiment of the present invention may include an apparatus configured to measure a person's movement via a plurality of sensors. The apparatus may include at least one first sensor configured to measure at least one rotational value during the movement. The apparatus may also include at least one second sensor configured to measure at least one linear value during the movement, and at least one third sensor configured to measure a force applied from a portion of the person's body. The at least one rotational value, linear value and force are used to generate a user interface display of the person's movement on an electronic device.

Another example embodiment of the present invention may include a method of measuring a person's movement via a plurality of sensors. The method may include measuring at least one rotational value during the movement via a first sensor. The method may also include measuring at least one linear value during the movement via a second sensor, measuring a force applied from a portion of the person's body via a third sensor, and generating a user interface display of the person's movement on an electronic device based on the at least one rotational value, linear value and force.

### BRIEF DESCRIPTION OF THE DRAWINGS

FIG. 1 illustrates a top view of a right handed golfers lead arm wearing the motion sensor device, according to an example embodiment of the present invention.

FIG. 2 illustrates a side view of a right handed quarterback in the motion of throwing a football wearing the motion sensor device, according to an example embodiment of the present invention.

FIG. 3 illustrates a diagram of components of the motion/force sensing device, according to an example embodiment of the present invention.

FIG. 4 illustrates the skeletal bones of a right hand of a person, first palm up and then palm down, wearing the motion sensor device, according to an example embodiment of the present invention.

FIG. 5 illustrates data characteristics of a force sensing element, according to an example embodiment of the present invention.

FIG. 6 illustrates other data characteristics of a force sensing element, according to an example embodiment of the present invention.

FIG. 7 illustrates a golfer at the top of his backswing referenced within a 3-D Cartesian coordinate diagram according to an example embodiment of the present invention.

FIG. 8 illustrates a display window of a smartphone or other handheld device displaying an example of the athletic analysis software, according to an example embodiment of the present invention.

FIG. 9 illustrates a computer readable medium that may be used to execute the application, according to an example embodiment of the present invention.

FIG. 10 illustrates a flow diagram according to an example method of the present invention.

### DETAILED DESCRIPTION OF THE INVENTION

It will be readily understood that the components of the present invention, as generally described and illustrated in the figures herein, may be arranged and designed in a wide variety of different configurations. Thus, the following detailed description of the embodiments of a method, apparatus, and system, as represented in the attached figures, is not intended to limit the scope of the invention as claimed, but is merely representative of selected embodiments of the invention.

The features, structures, or characteristics of the invention described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present invention. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

Wrist release is important to the accuracy of an athletes movements, especially in ball-related sports. The measurement of such a wrist cocking and releasing motion could be measured and analyzed by a device attached to the wrist. In the field of athletic motion monitoring it may be prudent to measure force, angular velocity, acceleration, rotational rate values, etc. of an athletes motion. For example, measuring an arm motion with its wrist cock and release motion.

Example embodiments of the present invention may include obtaining measurements from six-axes by using a sensing microchip(s) that includes gyroscope sensors and accelerometer sensors, and, one or more force sensing resistors that may be affixed to the player's wrist. Further components may include a microprocessor, a wireless transmitter and/or receiver, and a battery. Each of these components may be housed in a wristband that may easily be attached to the athletes wrist and worn to measure the overall athletic motion and timing of the cocking and releasing of the wrist during the athletic movement.

The data collected from such sensors are then processed by the microprocessor and transmitted to a computational device (i.e., computer, laptop, handheld mobile device (i.e., cell phone, smartphone, PDA, iPhone®). All values transmitted are then calculated and graphically displayed on the handheld device screen via a related software application that is installed on the device.

The widespread use of such products, such as, Blackberries® and Apple's iPhone® and iTouch® along with the popularity of the touchscreen “apps”, provides pocket computing integrated in the lives of millions worldwide. With the advent of such “smartphones” and PDA's it is unnecessary to further discuss the exact type of computational device that will be used to provide the calculation and application software tool.

Example embodiments may include a wrist (or ankle) worn device that an athlete would wear. The device would be capable of collecting all the movement data necessary to complete an accurate analysis of an athletic motion. In order to reduce costs associated with the wrist or ankle worn device, the data could then be transmitted via a wireless Bluetooth® or other wireless standard (IEEE 802.x) to a device that already possesses memory, a microprocessor, a display and is readily designed to store an application and execute a software program that may provide the user with the end result.

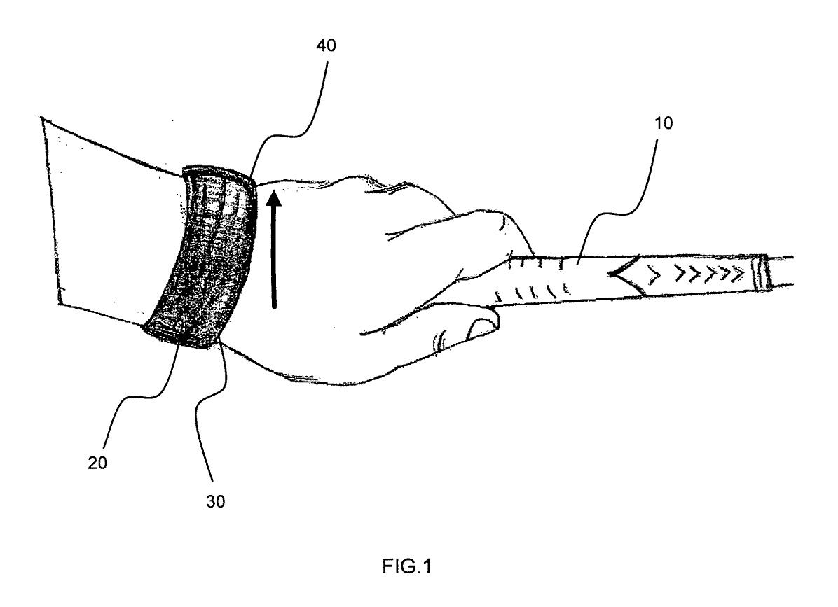

FIG. 1 illustrates an example of a golfer wearing a wrist bracelet according to example embodiments of the present invention. Referring to FIG. 1, a user wrist is illustrated wearing a wristband 20 while the user's hand is gripping a golf club 10. In this example, the golfer's hand is in a relatively neutral position, and is slightly cocked to the left relief space 30, which is the approximate position where the wristband 20 meets the inside portion of the golfer's wrist.

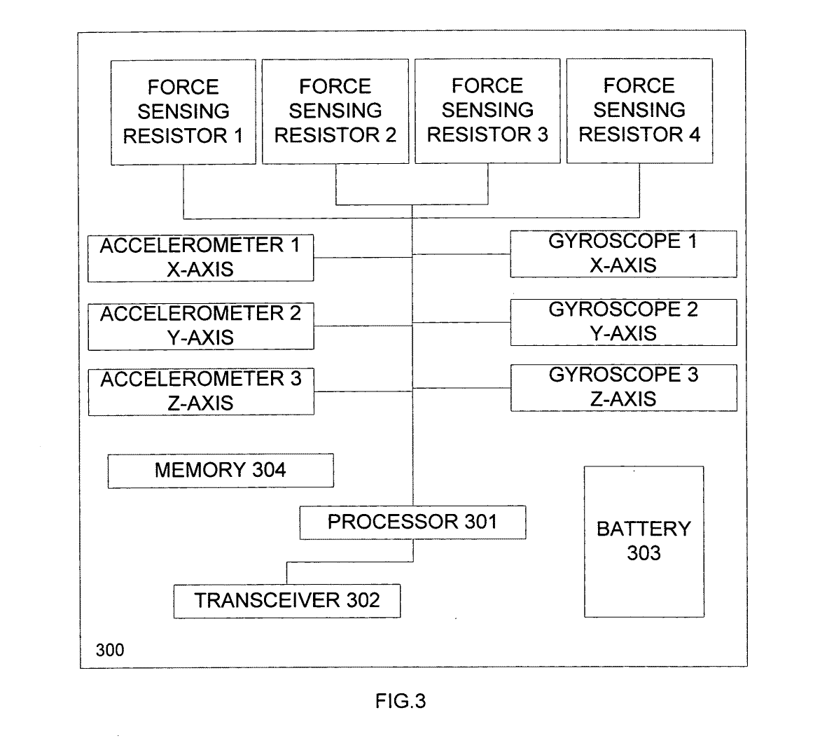

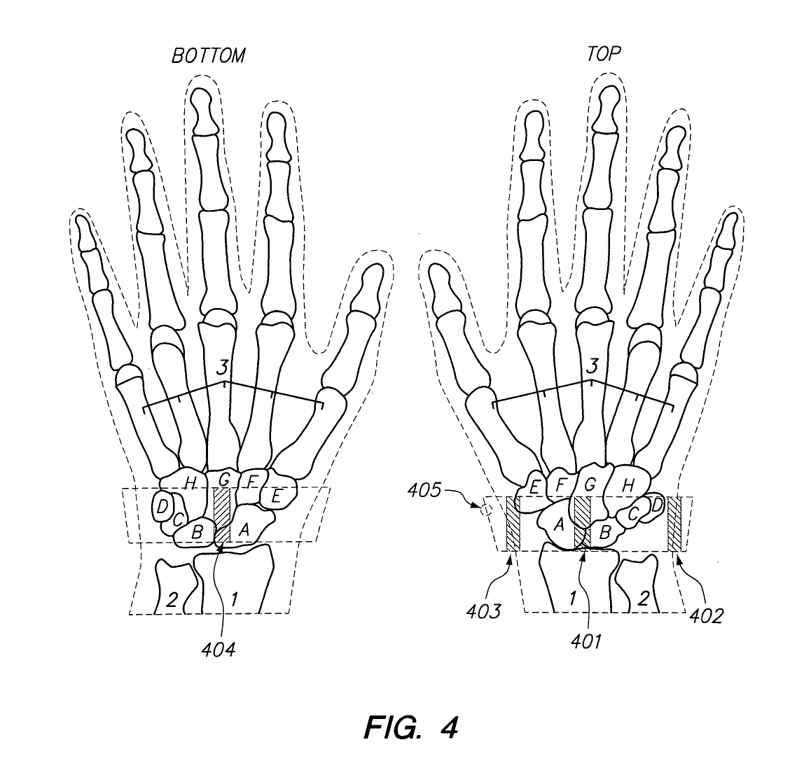

As may be observed from FIG. 1, there is slightly more “pinching” on the top side of wrist band near space 40, as indicated by the position of the wrist and the direction of the arrow. This is due to the user's wrist being angled slightly outwardly while holding the golf club 10. This “pinching” would engage one or more of the force sensing resistors (see force sensing resistors 1-4 of FIGS. 3 and 401-404 of FIG. 4) depending on the orientation of those sensors within the wristband 20. The engaging may cause an electrical signal to be initiated (i.e., a rise in conductance), and sent directly to a processor which would handle processing via the processor's connections in a flexible circuit board.

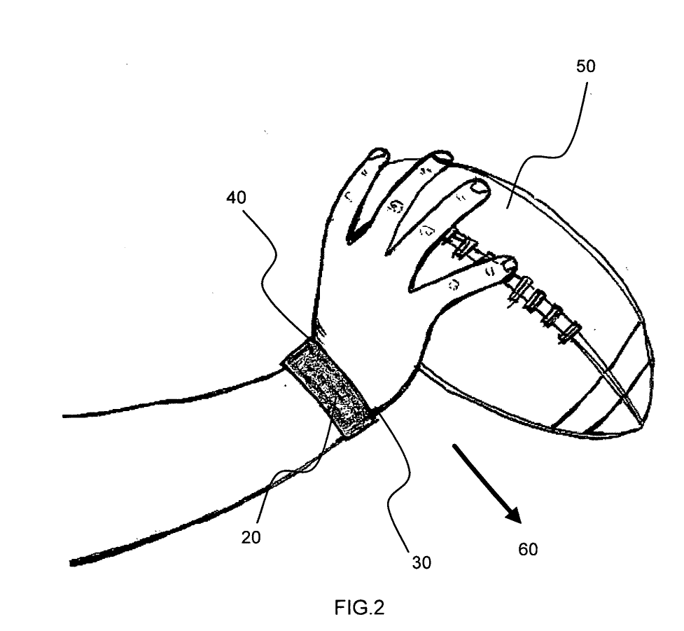

FIG. 2 illustrates another example of the wristband 20, according to example embodiments of the present invention. Referring to FIG. 2, the wristband 20 is illustrated as being attached to the wrist of a right-handed quarterback during the motion of throwing a football 50. There may be an increased “pinching” force near area 40. This “pinching” would engage one or more of the force sensing resistors (see force sensing resistors 1-4 of FIGS. 3 and 401-404 of FIG. 4) depending on the orientation of those sensors within the wristband 20. The engaging may cause an electrical signal to be initiated (i.e., a rise in conductance), and sent directly to a processor which would handle processing via the processor's connections in a flexible circuit board.

Referring to indicator 60, the arrow points to an area that signifies the direction of the intended motion. Referring again to FIG. 3, the accelerometers 1-3 are sensors which represent axes X, Y, and Z at any particular moment during the quarterback's movement of his wrist. These accelerometer sensors can detect the corresponding linear velocities in each of their respective directions (i.e., X, Y and Z). These data samples may then be communicated to the processor 301 of the motion detection and analyzing device 300, which may be part of the wristband 20.

Other data may be collected from the golfer or the quarterback's movements. For example, gyroscope sensors 1-3, which represent the X, Y, and Z axes, respectively, may be configured to detect the corresponding rotational positions and velocities at any particular moment during the movement of the athletes' arms and/or wrists. These signals may be obtained and stored in memory 304 and/or transferred directly to a computing device via transceiver 302. These signals may also be transferred to the processor 301 for processing via connections made in a flexible circuit board.

In FIG. 2, although a quarterback's throwing motion is depicted in this figure, the aforementioned principles would apply to a multitude of sports including sports using clubs or rackets, and sports which use repetitive wrist/arm motion. According to one example embodiment of the present invention, there are three different types of values collected, which include, force, linear velocity, and rotational orientation/velocity. The collecting of all three value types and the processing of these values would provide a detailed analysis of the user's athletic motion, and, in particular the motion of the athlete and his or her wrist movement during that particular motion.

FIG. 3 illustrates an example block diagram of the elements of the motion sensing device, according to example embodiments of the present invention. Referring to FIG. 3, there are four force sensing resistors 1-4. There may be additional sensors added to offer further force detection, however, in this example of FIG. 3, only four sensors are used to measure the force exerted from the athlete's wrist movements.

FIG. 4. illustrates the anatomy of a human's right hand along with the four force resistor sensors 401-404 according to an example embodiment of the present invention. Referring to FIG. 4, the skeletal bones of a right hand are shown from the top and bottom perspectives. Portions 1-3 represent the hand bones adjacent the carpel bones. The various proximal and distal carpal bones are listed A-H, proximal: A=Scaphoid, B=Lunate, C=Triquetral, D=Pisiform; distal: E=Trapezium, F=Trapezoid, G=Capitate, H=Hamate. The motion/force sensing wristband device is outlined on the wrist numbered as 405, and force sensors are the darkened shaded areas within the wristband and numbered 401-404. Sensors 401, 402, and 403 are not shown on the right hand palm-up (bottom) orientation.

One skilled in the art would recognize how these carpal bones and the muscle tissue that surrounds them would engage the force sensors 1-4 when the wrist is bent and moved. In one example, when the wrist is bent forward in a flicking motion to shoot a basketball, at a minimum, sensor 404 would be engaged due to a force applied to sensor 404 form the user's wrist. In another example, when the wristband is worn on a right handed quarterback, at a minimum, sensors 401 and 403 would be engaged when the quarterback is at the extent of his wind-up for a pass and then disengaged as he follows through with his pass, then, almost simultaneously, sensors 402 and 404 will engage as the quarterback puts the final finesse at the end of the passing motion.

In another example, when the wristband is worn on the left hand of a right-handed golfer, sensor 404 would be engaged at the address of a golf ball, as the golfer begins his backswing the force on sensor 403 is eased up and the position of the wrist and its force would shift onto sensor 402 at the top of his backswing. Next as the golfer follows through with his downswing, force is rapidly released from sensor 402 and almost all at once transferred to sensor 403 immediately preceding the moment of inertia. One skilled in the art can see that the principles embodied in these above-noted examples would be easily transferred to a multitude of other sports.

One example force sensor would be a tactile sensor made from piezo-resistive material. Referring to FIG. 4, the four force sensing resistors are placed along the top and bottom of the user's wrist. In this example of FIG. 4, the wristband is worn on the right hand, and the force sensor 401 is in direct proximity and centered between the carpal bones scaphoid-A and lunate-B, which are the proximate carpal bones on the bottom of the right hand wrist. Force sensor 402 is 90 degrees counter-clockwise from force sensor 401, and is in direct proximity to the pisiform-D carpal bone. Force sensor 403 is 180 degrees counter-clockwise from 401 and is in direct proximity and centered between the palm side of the scaphoid-A and lunate-B carpal bones. Force sensor 403 is 270 degrees counter-clockwise from 401 and is in direct proximity to the trapezium-E distal carpal bone.

According to one example embodiment, the depth location of the force sensors 401-404 within the body of the wristband may be positioned substantially near the extremity of the wristband material away from the user's wrist (i.e., near the top ring of the wristband). The depth that the force sensors 401-404 are located in the wristband may be designed such that the force sensors 401-404 will not be incidentally triggered with simple movements of the wrist that are not intended to trigger the force sensor. For example, if the movement of the wrist generates some force against all portions of the wrist, however, certain portions of the wrist may generate a greater force than other portions for a particular wrist movement. Therefore, the depth of the force sensors within the wristband should be limited to a depth that will not trigger force sensors that were not intended to be triggered for a particular wrist movement.

The accelerometer sensors 1-3 of FIG. 3 measure values of linear velocity along the X, Y and Z axes. In one example embodiment these three accelerometers would be a micro electro-mechanical system (MEMS) type of accelerometer. On the right side of the diagram are the gyroscope sensors which would measure the angular velocity along the X, Y and Z axes. In one example embodiment, these three gyroscopes 1-3 would be also be MEMS type gyroscopes.

Many different manufactures of MEMS motion sensing microchips exist, such as, the six-axis motion processing solution by InvenSense Inc®. This “package” of motion sensors includes a dual-axis gyroscope (X,Y) preset to a full-scale-range of ±500°/sec, a single-axis gyroscope (Z) preset to a full-scale range of ±2000°/sec, a triple analog to digital convertor with 16-bit sensor outputs through I2C or SPI interfaces, and a triple-axis (X,Y,Z) accelerometer with a programmable full-scale range of ±2 g, ±4 g, ±8 g and ±24 g.

Referring to FIG. 3, all of the sensors are coupled to the processor 301. Measurement values and data from all three different sensors are provided to the processor for processing. One skilled in the art would employ a proper type of processor, taking in to consideration the limited footprint available on the circuit board, power consumption, and adequate speed needed to undertake the proper task.

Also included is the transceiver 302, which would preferably be a low-power transceiver used to communicate with a handheld device such as an iPhone®, PDA or other type of user interface device. In one example embodiment of the invention, a wireless communications protocol such as Bluetooth or 802.x shall be used to transmit the data from the transceiver 302 to the handheld device (not shown). However there are a number of known standards and protocols that are appropriate for implementing a wireless data transmission that may also be used. The described invention is not restricted to using any particular protocol and/or handheld device or laptop computer.

If proximity of the display device is an issue, the movement measurement device 300 may connect to a base station both in a small piconet or femtonet proximity distance and/or a base station of a cellular network to communicate the processed data to the user end device (i.e, the mobile station handheld device). Certainly, the measurement device 300 would include a power supply or battery 303 in the wristband. One skilled in the art would implement any compact and/or rechargeable type of battery capable of providing output power from a designated voltage level. Also, one skilled in the art would take in to consideration the available footprint on the circuit board as well as longevity of the battery in designing the measurement device 300.

According to example embodiments of the invention, the necessary components of the detection device will be housed within a rubberized silicone wristband. The materials used for the housing may include weather proofing, durability, and flexibility to avoid water damage or cracking of the circuitry included inside. The wristband may have a hinge or flex point on one side and a clasp on the opposing side allowing for easy application and removal to the user's wrist. One skilled in the art would employ a rubber-based material or other flexible material.

The force sensors 1-4 may be placed within a few millimeters of the inner diameter of the wristband housing, so as not to impede the force sensing elements 1-4. Further, because proximity of the carpal bones to the force sensing resistors are tantamount to the sensors receiving the movement signals from the athletes motion, the wristband should be made in accommodating sizes taking into consideration the varying bone sizes of the athletes. For example, small, medium, and large, as well as x-large sizes may be produced.

It is inevitable that not all users of the wristband will fit into these four categories thus one of skill in the art may employ a snug-fitting highly flexible silicone cuff to bridge the gaps between the discrete sizes. The outer diameter of the wristband's cuff will fit against the inner diameter of the wristband, and the inner diameter of the wristband's cuff would fit snugly to the user's wrist.

The cuff may come in three to four different sizes within the four above-noted categories, allowing for the greatest diversity of users. Undoubtedly the use of a sizing cuff will impede the resistance to the force sensors, thus the application could include a program that adjusts the software parameters to allow for the sensitivity of the force sensors relative to the cuff size being used.

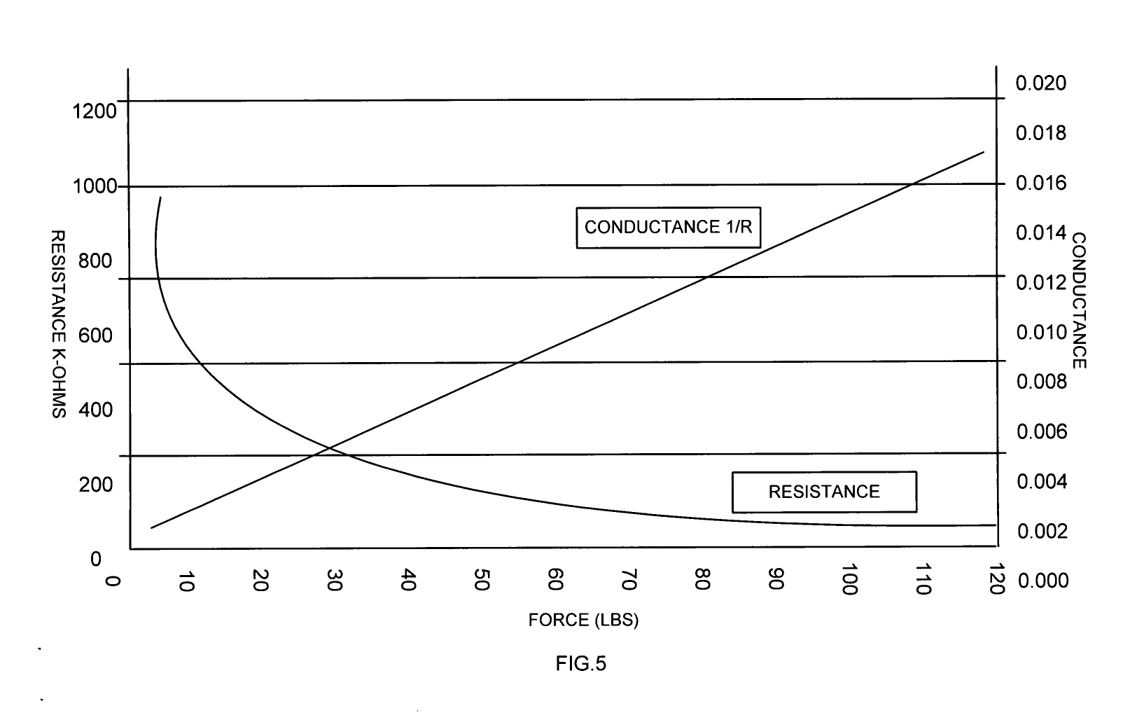

FIG. 5, depicts data characteristics of a force sensing element, according to an example embodiment of the present invention. Referring to FIG. 5, the graph illustrates the characteristics for a sample 100 pound (lb) sensor. The x-axis represents the force in lbs. The y-axis represents the resistance in kilo-ohms (K-ohms). The first line is substantially constant and represents conductance 1/R. The wavy line intermittently crosses a straight line that is used to show the substantial straightness of the conductance. The second line is the resistance and is shown to drop between 5 and 20 lbs. of force.

The conductance curve is relatively linear, and therefore useful during calibration. The single element force sensor (i.e., any or more of forces sensors 1-4) acts as a force sensing resistor in an electrical circuit. When the force sensor is unloaded, its resistance is very high. When a force is applied to the sensor, this resistance decreases. The resistance value can then be processed by connecting the sensing element to the processor 301 in FIG. 3 via a flexible circuit board. To integrate the force sensor into the device 300 it may be prudent to incorporate it into a force-to-voltage circuit. Calibration is necessary to convert the output of the force sensor into the appropriate units for measurement analyzing. Depending on the setup, an adjustment could then be done to increase or decrease the sensitivity of the force sensor.

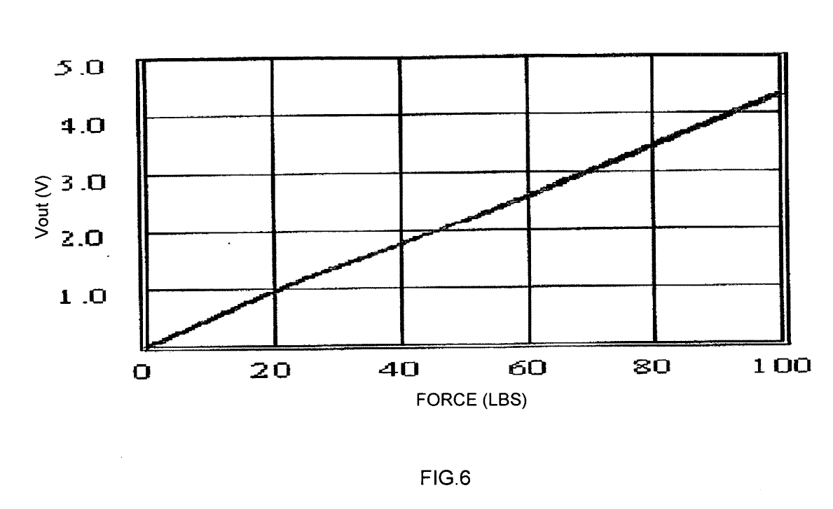

The graph in FIG. 6 illustrates a typical force sensor response. Referring to FIG. 6, a force is represented by the x-axis and a voltage output is represented by the y-axis. As the force increases, the output voltage also increases. In operation, a golfer at the top of his backswing referenced within a 3-D Cartesian coordinate diagram, the distances between two points, as defined by a Cartesian coordinate algorithm, can be calculated based on the following distance formulas. For example, The Euclidean distance between two points of the plane with Cartesian coordinates (x1,y1) and (x2,y2) is:

d=\sqrt{(x₂−x₁)²+(y₂−y₁)²}.

In the Cartesian version of the Pythagorean theorem three-dimensional space, the distance between points (x1,y1,z1) and (x2,y2,z2) is:

d=\sqrt{(x₂−x₁)²+(y₂−y₁)²+(z₂−z₁)²}.

Such a distance may be obtained by two consecutive applications of the Pythagorean theorem.

In one example embodiment, parameters of a golfer's swing could be measured by all three different types of sensors (accelerometer, gyroscope, and force sensor), then graphically displayed via a software application. The algorithms used to create the swing path and related diagrams would be based on a 3-D Cartesian coordinate system that would provide a full view of the golfer's swing.

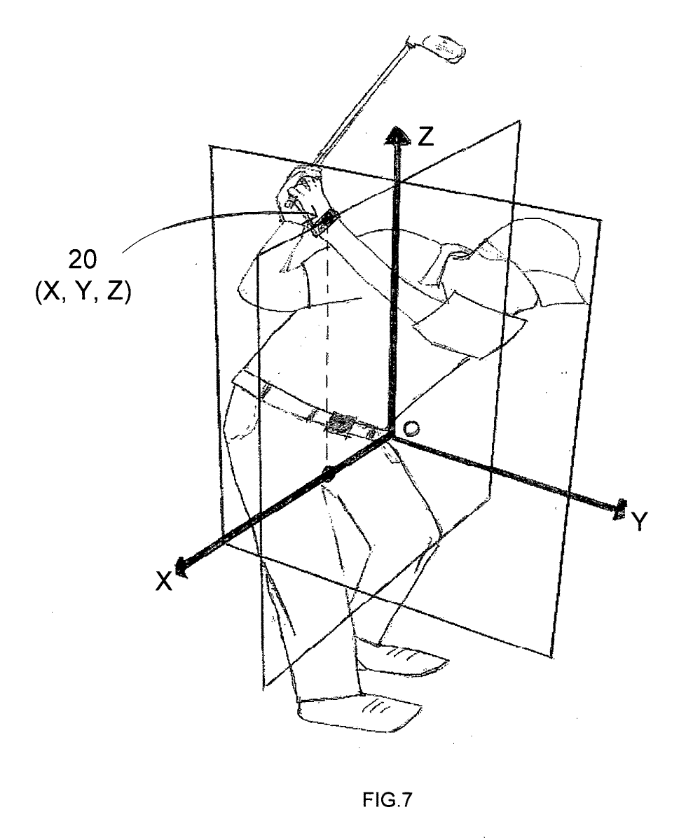

FIG. 7 illustrates a 3-D diagram of the golfer and his swing as measured from the analysis/training software, according to an example embodiment of the present invention. The measured values collected from the three types of sensors within the wristband could be graphically display.

Another set of algorithms derived from the data produced by the force sensing resistors (as illustrated in FIGS. 5 and 6) would need to be employed to track the position of wrist and the force of the wrist throughout the swinging motion in. The algorithms would track the force applied to each of the force sensors over time and the an analysis could be performed to determine sudden changes in the wrist movement that would be equated to faulty wrist behavior that user could attempt to eliminate on future attempted swinging motions.

Additional information may be included in the software application to take into consideration the specific golfer's set of clubs (i.e. type of club, club length, shaft type/flex, loft angle, and lie angle). This information would provide a more accurate application display that would display the golfer's exact equipment along with the measured movements. The known parameters from the user's equipment would be programmed into the software application to provide an accurate portrayal of the swing measured. For example, the length of the club could be calculated to provide a more accurate club speed movement.

As a result of including such additional parameter data in the application, further analysis and suggestions may be made to the golfer, such as, “swing down and through the line while releasing your wrist to produce a straighter shot.” Such improvements may include the golf club swing plane, club head-speed, club face-angle at moment of inertia, club face-pitch at moment of inertia, and club head-path prior or during moment of inertia.

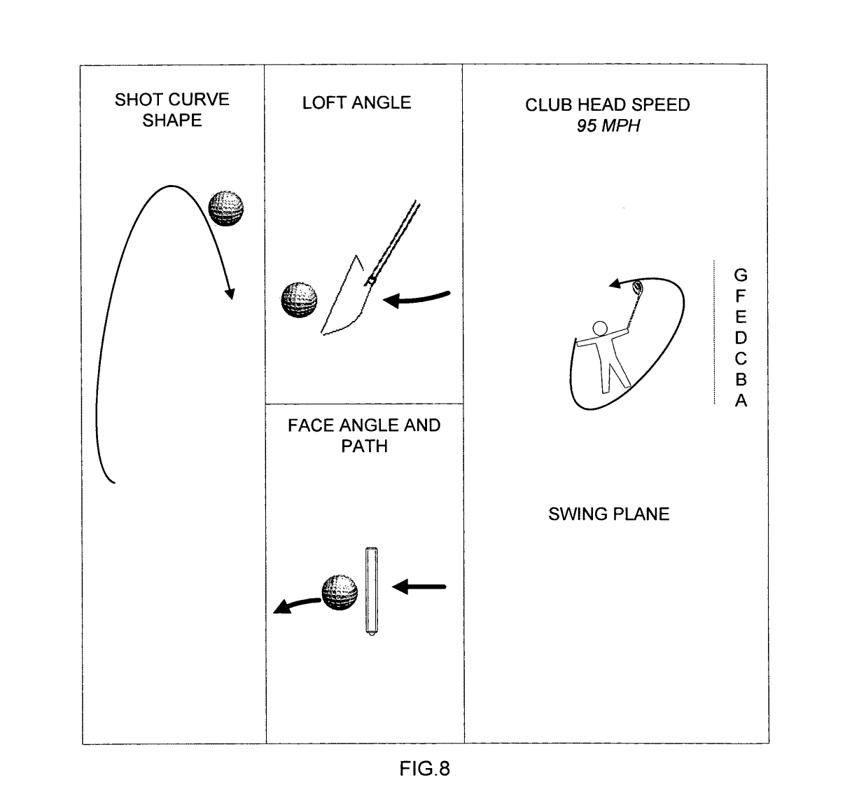

These example are illustrated in FIG. 8, which illustrates an example graphical user interface (GUI) display of the user application displayed on the user device (i.e., mobile phone “iPhone”®, pocket computer, etc). These animations may be displayed and recorded in real time, and later replayed for the user to review the outcome of his swing. The different attributes of the golfer's swing may be programmed into various animations (i.e. a golfer swinging his club, the plane of the swing shaded in a particular color, and, a club head passing through a golf ball with the path of the club head shaded in a particular color).

Additionally, numerical details of the club swing could be programmed for visual display alongside the animation (i.e. club head speed, shot shape, loft angle, fall angle, path, tempo, angle of pitch at impact, angle of club face relative to the corresponding line of a golf ball, swing plane etc.). Some of these example parameters and views are illustrated in the GUI display of FIG. 8.

In one example of the operation of the present invention, a golfer would start his session by wearing the wristband, turning it on, wirelessly “pair” it with an smartphone (iPhone®) or other PDA, and execute the corresponding application on the smartphone/PDA. Next, the software would prompt the user to establish a neutral position for his wrist. This neutral position may be with his arm by his side pointing down (see FIG. 7, area marked as “O” for origin). Then the user is prompted by the software application to “set” this position as the origin.

This origin will be used as a baseline or zero point for all 3-D Cartesian coordinate system algorithms thereafter within the software. The user can then select any club he wishes from his bag, and accordingly select it within the software application. The golfer may then set a golf ball into play (teeing up the ball), and address the ball by inputting the ball's position. The software application could be further programmed to detect and “learn” the user's regular pre-swing routine. This is commonly a series of body waggles and/or arm/wrist movements followed by the golfer squaring his club face to the ball, followed by a brief pause and the start of the golfer's backswing. Once the software has learned the user's routine it may then detect accurately the user's address position (moment just prior to the start of the backswing), and accordingly set that point (club face alignment as it relates to the golf ball) as the ideal return position or moment of inertia position.

The software application would have the capability to successfully calculate and produce any swing analysis discrepancies that may arise when the golf ball is lying on a slope, or, whether the ball is above the golfer's feet or below it, etc. In summary, the result would be an accurate portrayal of the golfer's stroke, each and every time, no matter what conditions are presented on the course.

Example embodiments may further include the use of GPS capabilities within the smartphone/PDA, to provide golf course layout, yardages, and current yardage to each hole during course play. Current phones offer GPS capabilities, such as, the iPhone®. The employment of such capabilities would be integrated within the software application. Further applications may include a suggestion feature that offers an appropriate club for the golfer to use for a particular hole on the course. Further suggestions may include, a suggested ball flight shape and speed to swing based on integrated GPS golf course information and the library of previous swing analysis recorded by the user.

Each stroke taken in a round of golf could be recorded and stored with the GPS-derived map of the course for the user to review instantly or at a later time. This could provide a library of courses frequented by the user along with a library of each stroke along with its outcome throughout the user's season for repeated use by the user. The handheld device's microphone capabilities could also be used to detect the current wind speed by the noise level associated with the wind, and, such data could automatically be incorporated into the swing analysis. Also, if the smartphone has a built-in digital compass then direction of the wind could be accurately determined. This feature would be of particular interest for golfers.

The training/analysis software may also provide a brief setup tip to the user based on which shot shape is recommended (i.e., the golfers foot position, ball position in the golfer's stance, and grip position on the golf club). The user may then place the handheld device in his pocket and take a few practice swings with his recommended set-up. Once the proper club head speed, swing plane, club path, face angle, and loft angle have been detected by the software application during the user's practice stroke, the handheld device will alert the user via a vibration or audible tone. The user will then be impelled to remember that same feeling and take his real stroke.

Professional athletes may be asked to train with the wristband for a few sessions. For instance, a professional golfer may be asked to play a few rounds of golf using the wristband. After all data has been collected, the data may be incorporated into a program to illustrate the important parameters of what makes that golfer successful and efficient. Such a set of data may be included in a training regime within the analysis software application. The program would have the ability to detect the differences between the user's current swing and the swing of a professional golfer, and offer a training session on how to improve their game.

Pre-recorded video tips from professional golfers may also be referenced to illustrate the types of errors recognized by the program and the pre-recorded solution made by the professional may be automatically invoked based on the type of golf swing measured by the application. As an example, after the user performs badly on a shot, the software application will prompt the user to watch a short clip from a professional golfer, where the golfer acknowledge the problem of the users swing and then offers a demonstration on how to correct that particular problem. This would be easy to match with the user's swing since the outcome of the various types of “poor” shots are usually within common parameters of the mechanics of the user's swing. For instance, a bad “slice” may be one of two, or, a combination of the club face being open during impact, or, the club path sweeping across the ball in an out-to-in pattern. Those undesirable parameters may be programmed into the software for easy detection. Once these parameters have been recognized, the software can then prompt the user to watch a video clip matched that type of problem and offering tips to correct the problem.

The software application may be available for sale via a subscription method. This subscription service allows continued revenue for the producer of the device and method. With this continued revenue the producer of the device and method may continue to keep the content of the analysis software updated. For example, periodically updating and adding to the pre-recorded video tips from the professionals. Also, updating course information and adding in new courses as they are built.

In a further embodiment, the wristband may include an analysis method that may be used in the telecasting of sporting events. For instance, professional golfers may wear the wristband and via wireless protocols the motion of their swing may be communicated to off-site computers, and, broadcasted to the viewers of the sporting event. Hence a more accurate commentary can be made on the swing motion and outcome of the shot, and comparisons can be made versus other golfers or previous performances.

The user may desire to have the device present their training sessions to scouts via an Internet database. What results is the ability for athletic scouts to virtually view the athletes without ever visiting them. Potential gifted athletes may be discovered based on the motions they prerecorded throughout their training sessions versus known great performances and know preferred athletic mechanics. In addition, the scouts may contact the athletes and tell them their deficiencies so that the user can practice and record improvement sessions to prove that they are improved and ready for recruitment.

Although the sports of golf and football were used as examples in this disclosure, one of ordinary skill in the art will be able to transpose the same principles into a multitude of different sports including baseball, hockey, tennis, basketball, and even Olympic sports such as javelin throwing, discus, pole vault, etc. Further, the use of a handheld device, smartphone, and PDA were commonly referred to in these embodiments, however one skilled in the art could perceivably employ a diverse range of computational machines (i.e. laptop computers, desktop computers, and video game consoles). Hence the preferred embodiments have been included in conjunction with the present invention and method; however a plural of details, improvements, and modifications may become recognized by one skilled in the art without departing from the spirit and scope of the invention.

Therefore it is to be ascertained that the spirit and scope of the present invention not be limited or confined to the above embodiments, but be detailed in harmony with the following claims and their equivalents.

The operations of a method or algorithm described in connection with the embodiments disclosed herein may be embodied directly in hardware, in a computer program executed by a processor, or in a combination of the two. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

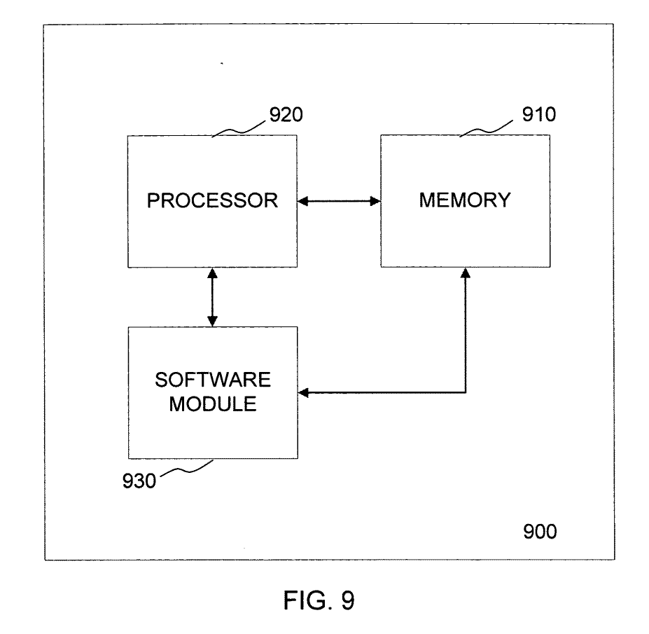

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example FIG. 9 illustrates an example network element 900, which may represent any of the above-described computational devices used to display the results of the application program.

As illustrated in FIG. 9, a memory 910 and a processor 920 may be discrete components of the network entity 900 that are used to execute an application or set of operations. The application may be coded in software in a computer language understood by the processor 920, and stored in a computer readable medium, such as, the memory 910. Furthermore, a software module 930 may be another discrete entity that is part of the network entity 900, and which contains software instructions that may be executed by the processor 920. In addition to the above noted components of the network entity 900, the network entity 900 may also have a transmitter and receiver pair configured to receive and transmit communication signals (not shown).

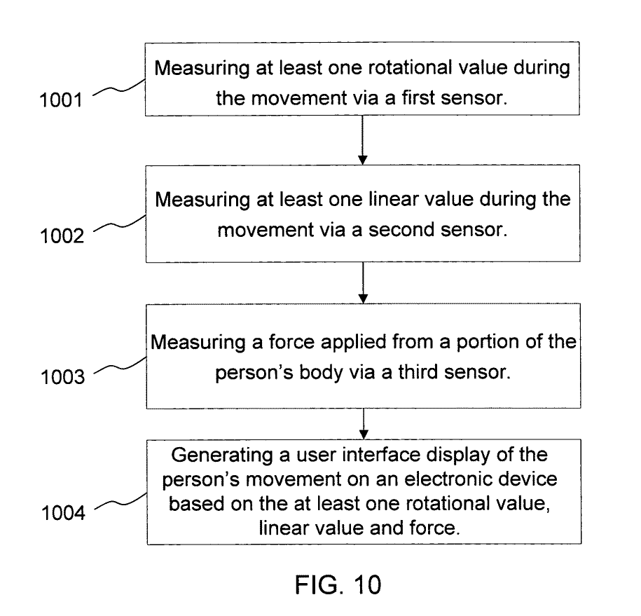

One example method of the present invention may include a method of measuring a person's movement via a plurality of sensors, as illustrated in FIG. 10. The method may include measuring at least one rotational value during the movement via a first sensor, at operation 1001. The method may also include measuring at least one linear value during the movement via a second sensor, at operation 1002, and measuring a force applied from a portion of the person's body via a third sensor, at operation 1003. Another operation may include generating a user interface display of the person's movement on an electronic device based on the at least one rotational value, linear value and force, at operation 1004.

While preferred embodiments of the present invention have been described, it is to be understood that the embodiments described are illustrative only and the scope of the invention is to be defined solely by the appended claims when considered with a full range of equivalents and modifications (e.g., protocols, hardware devices, software platforms etc.) thereto.

What is claimed is:

1. An apparatus configured to measure a person's movement via a plurality of sensors, the apparatus comprising:
at least one first sensor configured to measure at least one rotational value during the movement;
at least one second sensor configured to measure at least one linear value during the movement; and
at least one third sensor configured to measure a force applied from a portion of the person's body,
wherein the at least one rotational value, linear value and force are used to generate a user interface display of the person's movement on an electronic device.

2. The apparatus of claim 1, wherein the first sensor is a gyroscope sensor, the second sensor is an accelerometer sensor and the third sensor is a force sensor.

3. The apparatus of claim 2, wherein the at least one force sensor comprises four force sensors.

4. The apparatus of claim 3, wherein each of the four force sensors are attached to a wristband worn by the person and wherein at least one of the four force sensors are positioned contiguous with at least two carpal bones in the person's wrist.

5. The apparatus of claim 1, further comprising:
a transmitter configured to transmit data based on the at least one rotational value, linear value and force to a mobile station to generate the user interface display of the person's movement.

6. The apparatus of claim 5, wherein the mobile station is at least one of a Blackberry®, iPhone® or other type of smartphone device.

7. The apparatus of claim 5, wherein the transmitted data is used to generate a three-dimensional model of the person's movement.

8. The apparatus of claim 1, further comprising:
a processor configured to compute the user's movement based on the at least one rotational value, linear value and force; and
a transmitter configured to transmit the computed user's movement to a mobile station.

9. A method of measuring a person's movement via a plurality of sensors, the method comprising:
measuring at least one rotational value during the movement via a first sensor;
measuring at least one linear value during the movement via a second sensor;
measuring a force applied from a portion of the person's body via a third sensor; and
generating a user interface display of the person's movement on an electronic device based on the at least one rotational value, linear value and force.

10. The method of claim 9, wherein the first sensor is a gyroscope sensor, the second sensor is an accelerometer sensor and the third sensor is a force sensor.

11. The method of claim 10, wherein the at least one force sensor comprises four force sensors.

12. The method of claim 11, wherein each of the four force sensors are attached to a wristband worn by the person and wherein the each of the four force sensors are positioned contiguous with at least two carpal bones in the person's wrist.

13. The method of claim 9, further comprising:
transmitting data based on the at least one rotational value, linear value and force to a mobile station to generate the user interface display of the person's movement.

14. The method of claim 13, wherein the mobile station is at least one of a Blackberry®, iPhone® or other data processing device.

15. The method of claim 14, wherein the transmitted data is used to generate a three-dimensional model of the person's movement.

16. The method of claim 9, further comprising:
computing the user's movement based on the at least one rotational value, linear value and force; and
transmitting the computed user's movement to a mobile station.

17. A computer readable storage medium comprising a computer program that when executed causes a processor to perform a method of measuring a person's movement via a plurality of sensors, the processor performing:
obtaining at least one rotational value during the movement via a first sensor;
obtaining at least one linear value during the movement via a second sensor;
obtaining a force applied from a portion of the person's body via a third sensor; and
generating a user interface display of the person's movement on an electronic device based on the at least one rotational value, linear value and force.

18. The computer program of claim 17, wherein the processor is further configured to perform:
estimating a current user position via a GPS position estimate; and
providing the user with a map of an area adjacent the user and incorporating the map into the user interface display.

19. The computer program of claim 18, wherein the user interface display is provided to a user mobile station, and wherein a wind speed of the user's location is measured based on a sound measurement observed from the user's mobile station microphone.

20. The computer program of claim 19, wherein prerecorded information advice is offered to the user via the mobile station based on at least one of the GPS position estimate of the user, the measured wind speed and a swing motion performed by the user.
